(19) United States
(12) Patent Application Publication
(10) Pub. No.: US 2011/0300504 A1
(43) Pub. Date: Dec. 8, 2011

(54) METHOD AND LASER PROCESSING DEVICE FOR PROCESSING TISSUE

(75) Inventor: Anton KASENBACHER, Traunstein (DE)

(73) Assignees: Lumera Laser GmbH, Kaiserslautern (DE); Dr. Anton Kasenbacher, Traunstein (DE)

(21) Appl. No.: 13/097,834

(22) Filed: Apr. 29, 2011

Related U.S. Application Data
(63) Continuation of application No. PCT/EP2009/006022, filed on Aug. 19, 2009.

(30) Foreign Application Priority Data
Oct. 30, 2008 (DE) 10 2008 053 964.3

(51) Int. Cl. A61C 3/00; A61B 18/20

(52) U.S. Cl. 433/29; 606/2; 606/11

(57) ABSTRACT

Method and laser processing device to process tissue. In a general aspect, the method to process tissue may include applying a photosensitizer into an area surrounding a region of the tissue to be processed, and irradiating the region of the tissue to be processed with the pulsed processing laser beam, the laser beam emitting laser pulses with a temporal full width at half maximum in a range between about 100 femtosecond and about 1 nanosecond. In another general aspect, the laser processing device to process tissue may include a laser radiation source to provide a pulsed processing laser beam providing emitting laser pulses, a laser beam decoupling unit to decouple the laser beam towards a region of the tissue to be processed, and an output device to output a photosensitizer in a direction of an area surrounding the region of the tissue to be processed, the output device being connected to the decoupling unit.

### CROSS-REFERENCE TO RELATED APPLICATIONS

This application is a continuation application, under 35 U.S.C. Section 111(a), of PCT International Application No. PCT/EP2009/006022, filed Aug. 19, 2009, which claimed priority to German Application No. DE 10 2008 053 964.3, filed Oct. 30, 2008, the disclosures of which are incorporated herein in its entirety.

### BACKGROUND

1. Field

The invention relates to a method and a laser processing device for processing tissue, and more particularly to a method for instantaneous diagnosis and processing of biological tissue, and to a laser processing device for automatic and objective diagnosis and processing of biological tissue.

2. Description of the Related Art

One example of the field of application of the invention is dentistry. In dentistry, a laser processing method and a corresponding laser processing device can be used instead of a mechanical drill for the ablation and removal of tooth material, particularly carious tooth material. However, the invention can likewise be applied to other types of biological tissues, such as hard tissue, soft tissue, and tissue fluids. By way of another example, ophthalmology can be another potential field of application.

In dentistry, in particular in caries therapy, a substantial goal has been to, completely or at least partially, replace the conventional X-ray technology for diagnostic and conventional mechanical drilling equipments each with a virtual monochromatic (LED) and/or exact monochromatic radiation sources (lasers). The mutagenic and carcinogenic potential of X-radiation are sufficiently well known in medical field so that it has been always necessary to act under the basic guideline of radiation protection, the ALARA principle (As Low As Reasonably Achievable). An ideal alternative would be an analysis without X-radiation during treatment, if possible. As to a processing of oral tissue structures, to date the conventional “drill” is used in dentistry because of its universality in conjunction with low investment costs despite of its considerable potential for thermo-mechanical damage (frictional heat, cracks, shockwaves) coupled with the resulting unavoidably pain. However, there is still no “smart” device for simultaneous and objective detection of pathological structures (e.g., caries) and therapy (e.g., cavitation preparation) with auto self-limiting stop accompanied by maximum bio-safety. All of these undesirable effects can be avoided by using a combined diagnostic and laser processing device.

In the course of recent years, a series of laser systems for dentistry have been tested. However, in many cases, either undesired thermal or other side effects have been observed, or the removal efficiency was inadequate. This applied especially to laser systems operating on the basis of pulsed laser beam sources with pulse widths ranging from nano to microseconds. For example, such lasers can be excimer lasers with wavelengths in the ultraviolet range or Er:YSGG (λ=2.7 μm) or Er:YAG lasers (λ=2.94 μm) in the infrared wavelength range. In addition, none of these systems is capable of performing bio-safe detection and therapy.

A substantial advancement was achieved after the introduction of short-pulse laser systems in the picosecond (ps) or femtosecond (fs) range and wavelengths in the visible or near infrared spectral range. First experimental studies indicated that these systems make it possible to achieve high quality dental ablation results with the efficiency at least equal to the performance of a mechanical turbine.

U.S. Pat. No. 5,720,894 describes a method and a device for material ablation by means of a pulsed laser beam source. The ablation parameters to be selected for wavelength, pulse width, energy and repetition rate of the laser pulses are indicated mainly just in reference to the task concerned. Here, each laser pulse is intended to interact with a thin surface portion of the material such that plasma is formed in the focal position of the laser beam. The cited parameters of the laser beam are indicated with a relatively wide range amounting up to 50 mJ or relative to the surface area, up to 15 J/cm². However, particularly when more than three photons are involved, the risk was that such a high pulse energy involving very short laser pulses where the values attained as to power or intensity in the maximum pulse, harmful collateral effects may be materialized due to non-linear processes such as multi-photon ionization. The risk is especially notable when the powerful peak pulses (of a few TW/cm²) that water molecule ionization occurs (ionization energy Eᵢₒₙ=6.5 eV) with fatal collateral effects (i.e. DNA damage and the formation of cavitation bubbles with subsequent unavoidable sonoluminescent fusion in a spectral bandwidth with a range from the ultraviolet (UV) to the radiographic range) as far as into the X-ray region.

### SUMMARY

It has been realized that what is needed in order to solve such limitations is to provide a device and a method for processing biological tissue, which may assure efficient tissue lasering while avoiding or minimizing the damaging effects of tissues being irradiated and of the immediate ambience. Also, additional flexibility in selecting the lasering wavelength may be achieved.

The embodiments of the present disclosure can be implemented to realize one or more of the following advantages. For example, the embodiments can be implemented to provide a comparatively low equipment outlay for processing a tissue so that the costs of the laser processing device can be kept within limits. In addition, the embodiments may also be implemented to process a tissue while i) instantaneously ensuring an objective diagnosis and efficient processing of a tissue; ii) avoiding or minimizing damaging effects on the diagnosed and processed tissue; and iii) enabling additional flexibility in the selection of the laser wavelength, laser pulse duration, and laser intensity.

In one general aspect, a method for processing a tissue, wherein a pulsed processing laser beam is provided in a processing mode, the method may include applying a photosensitizer into an area surrounding a region of the tissue to be processed, and irradiating the region of the tissue to be processed with the pulsed processing laser beam, the laser beam emitting laser pulses with a temporal full width at half maximum in a range between about 100 femtosecond and about 1 nanosecond.

In another general aspect of the present disclosure, a method for processing a tissue, wherein a pulsed laser beam is provided in a processing mode, the method may include irradiating a region of the tissue to be processed with the pulsed processing laser beam, the laser beam emitting laser pulses with a temporal full width at half maximum in a range between about 100 femtosecond and about 1 nanosecond, the laser beam having a laser pulse wavelength in a range between about 100 nm and about 10.6 μm, and the laser beam having an energy density per pulse in a range below 7.5 J/cm².

In another general aspect of the present disclosure, a laser processing device to process a tissue may include a laser radiation source to provide a pulsed processing laser beam emitting laser pulses, a laser beam decoupling unit to decouple the laser beam towards a region of the tissue to be processed, and an output device to output a photosensitizer in a direction of an area surrounding the region of the tissue to be processed, the output device being connected to the decoupling unit.

In another general aspect of the present disclosure, a laser processing device to process a tissue may include a laser radiation source to provide a pulsed processing laser beam emitting laser pulses, a decoupling unit to decouple the laser beam towards a region of the tissue to be processed, and focusing means for focusing the laser beam, the laser beam having a wavelength of the laser pulses in a range between about 100 nm and about 10.6 μm, a temporal full width at half maximum of the laser pulses in a range between about 100 fs and about 1 ns, and an energy density of the laser pulses on a surface of the tissue in a range below 7.5 J/cm² per pulse.

A first insight of one embodiment of the present disclosure consists in that when ablating biological tissue with a laser beam it is not mandatory for the laser beam to be applied to the tissue itself. Instead, the laser beam can advantageously be absorbed by a substance, as a consequence of the absorption acts essentially as a source of free or quasi-free electrons, and in such a way transmits the absorbed energy to the material to be removed. A so-called photosensitizer can be used most efficiently as to the substance.

Photosensitizers constitute chemical photosensitive compounds that enter into a photochemical reaction after the absorption of a light quantum. The activation of a photosensitizer can be performed by laser light of suitable wavelength and adequate intensity, the absorption of light firstly exciting the photosensitizer in a relatively short-lived singlet state that thereafter crosses to a more stable (long-lived) triplet state. This excited state can then, for example, react directly with the material to be removed.

A further insight of the present disclosure consists in making use for a diagnosis, which is, if appropriate, to be carried out together with a processing method, of a marker that visualizes regions that are to be processed or to be ablated, the marker likewise possibly being a photosensitizer and/or being capable of excitation by radiation of a laser or a light-emitting diode (LED) continuously or in pulsed fashion, with a suitable wavelength, duration and intensity. It can in particular be provided to use one and the same photosensitizer for the ablation and also for the diagnosis.

A further insight of the present disclosure consists in surrounding a region of a tissue that is to be processed or ablated by means of a laser beam with a sheath, and integrating a suction channel system inside said sheath and a laser beam decoupling unit connected therewith.

There are various possibilities with regard to the selection of the photosensitizer to be used and to the laser radiation source to be used for the ablation and/or the diagnosis. In accordance with the embodiments presented here, the photosensitizer and/or the wavelength of the laser pulses may be selected in such a way that the laser beam is absorbed at least partially by a single photon absorption in the photosensitizer, the absorption being in the vicinity of an absorption maximum of the photosensitizer. This embodiment may be, therefore, assigned to linear optics. In accordance with another embodiment, which may be assigned to nonlinear optics, the photosensitizer and/or the wavelength of the laser pulses may be selected in such a way that the laser beam is absorbed at least partially by a two photon absorption in the photosensitizer, the absorption being in the vicinity of an absorption maximum of the photosensitizer. In accordance with another embodiment, which may be likewise assigned to nonlinear optics, the photosensitizer and/or the wavelength of the laser pulses may be selected in such a way that the laser beam is at least partially absorbed by a multi-photon absorption in the photosensitizer, the number of the absorbed photons being equal to or greater than 2, and the absorption being in the vicinity of an absorption maximum of the photosensitizer.

In accordance with one embodiment, a repetition rate of the laser pulses may be set in a range between 1 Hz and 10 MHz, this range also encompassing each incremental intermediate value, and the increment being 1 Hz. In this case, it can also be provided that the laser pulses are generated in the form of bursts each having a prescribed number of laser pulses and that, for example, a prescribed number of bursts (for example, one burst) is applied to each region to be processed on the tissue to be ablated. The laser pulses can also have a pulse peak intensity varying in a prescribed way within a burst. It is advantageous when no sort of undesired prepulses or postpulses or background intensities and offset intensities occur during or after the bursts.

In accordance with one embodiment, the method may be used for the ablation or removal of tooth material, in particular carious tooth material. This may offer an advantageous field of application to the extent that carious tooth material is known to have a porous structure caused by bacterial attack. The photosensitizer can penetrate into this porous structure so that it is to some extent embedded in the carious tooth material to be removed, and need not—as required in other applications—be applied to a surface of a tissue material to be removed.

During the processing of biological tissue with a short pulse laser such as a picosecond or femtosecond laser within a thin surface layer a microplasma may be produced at the focus of the processing laser beam and decays after each individual pulse in the course of a time period in the nanosecond to microsecond range. During the ablation operation, the biological tissue may not be ionized per interaction of the laser photons with the quasi-free electrons, but minimally fragmented invasively in a thermomechanical fashion. A goal consists in generating the microplasma in the threshold region, that is to say always less than or equal to the critical electron density (at laser wavelength 1064 nm: 1.03×10²¹ electrons/cm³). This may provide the ablation operation to be carried out with the greatest possible medical and biological compatibility and the avoidance of undesired side effects. In particular, plasma temperatures of greater than or equal to 5800 K (temperature of the surface of the sun) accompanied by UV rays and multi-photon ionization may be avoided, in order thus to achieve the nonionization of water molecules present in the tissue. Furthermore, with regard to limiting the thermal and mechanical effects it may be advantageous when the pulse length of the laser pulses is in a range of approximately 100 fs−100 ps, in particular at approximately 10 ps. The inventive combination of an indirect energy input by means of photosensitizers and the use of picosecond laser pulses then may lead to maximum biological and medical compatibility of the treatment. Particularly with regard to stress relaxation, the picosecond laser pulses effect an optical penetration depth in such a way that no sort of shockwaves can propagate, and so the treatment can be carried out without pain.

In accordance with one embodiment, the energy density of the laser pulses may be set in a range below 7.5 J/cm². In particular the energy of the laser pulses can be set in a range below 100 μJ, and the focus of the processing laser beam on a surface of the tissue and/or the photosensitizer can be set with a focal diameter in a range between 1 μm and 100 μm, it being possible to achieve the range of 1 μm to 5 μm by means of special optics. These measures may ensure that the plasma density is always below the critical electron density. The use of a photosensitizer moreover may permit the use for the ablation of the biological tissue of an energy density of the laser pulses that is substantially below the above-named value, for example below 1.0 J/cm². This means that given appropriate energies of laser pulses it is possible to use conventional focal spot sizes, and there is no need for additional expenditure with regard to the focusing optics. However, it can also mean that given stronger focusing a laser radiation source with lower laser pulse energies can, if appropriate, be used, for example a laser radiation source that consists only of a laser oscillator without the use of a downstream optical amplifier. The expectable total costs of a laser processing device for carrying out the inventive method could thereby be reduced under some circumstances. Specifically, a commercial laser oscillator such as, for example, a solid state laser oscillator, in particular a picosecond laser oscillator, then suffices as laser radiation source. In order to be able to provide the most compact laser radiation source possible, it may also be, for example, conceivable to use a laser diode or a multiple arrangement (array) of laser diodes, in particular as picosecond lasers.

In general, the processing laser beam may be used to process a specific surface region of the biological tissue in order that, in accordance with a further embodiment, the laser beam may be scanned in a suitable way over this surface region. In this context, it can prove to be advantageous in addition when the processing laser beam has a substantially rectangular (also called top hat) beam profile. It can then be provided in the scanning operation that exactly one laser pulse is applied to each sub-region covered by the focus of the processing laser beam. This measure can also be provided independently of the presence of a top hat profile, for example, by stipulating that during the scanning operation, mutually adjacent sub regions, which are each covered by a laser pulse, have a mutual spatial overlap whose surface area is smaller than half or smaller than another fraction of the surface area of a sub-region. Even given the presence of a Gaussian profile as “laser beam cross section”, it is possible in this way for essentially only a single laser pulse to be applied to a sub-region covered by the focus of the processing laser beam.

In accordance with a further embodiment, it can be provided that the spatial position of the focus is constantly controlled to be on the surface of the region during processing. As is elaborated further below in more detail, this can be performed by an autofocus unit in a wide variety of embodiments.

In accordance with a further embodiment, it can be provided that before application of the photosensitizer, the region to be processed is determined by applying to the tissue a marker that may assume a characteristic coloration in contact with a specific tissue type, in particular damaged tissue, or that exhibits another detectable response, for example after excitation by coherent or incoherent electromagnetic radiation. In this case, the marker can likewise be supplied by a photosensitizer that thus constitutes a diagnostic photosensitizer, while the photosensitizer used for the ablation can be denoted as an ablation photosensitizer. The marker can, however, likewise be formed by another commercial marker which has no photosensitizer properties. In contrast, by way of example, the ablation photosensitizer is such that it has no marker properties, that is to say acts in a non-staining fashion upon contact with various tissue types.

In accordance with a further embodiment, it is also possible to use a photosensitizer that is firstly used as a marker and subsequently is used for the ablation. This could simplify the treatment in that it is only those regions of the photosensitizer that exhibit a color change, or exhibit another type of corresponding reaction during diagnosis, that are irradiated with the laser for the subsequent ablation. In other words, a photosensitizer that is identical to the photosensitizer used for the later ablation is used for the diagnosis. The diagnosis is then based, for example, on the same absorption properties of the photosensitizer as the later ablation.

In accordance with a further embodiment, the marker or photosensitizer used for the diagnosis and which is intended to mark or indicate damaged tissue can have additional properties. It can, for example, act as an acid (pH) indicator and/or as a bacteria and/or virus and/or tumor cell indicator, that is to say is able to indicate, by a specific response such as a color change or fluorescence radiation, the height of a pH value of an adjacent tissue, or the extent to which the tissue is infected with bacteria and/or viruses and/or tumor cells. The photosensitizer can also be designed so that it can act as a so-called 3D indicator, that is to say it is able to reflect or retroreflect the light pulses in such a way that they can be imaged on a detector, and a three-dimensional topography of the surface covered by the photosensitizer can be determined by determining the transit time of the light pulses emitted and retroreflected by a specific point. This can be used to particular advantage in a situation in which it has previously been established by means of one and the same photosensitizer that damaged tissue is no longer present, and therefore there is no need for further ablation operations. A cavity produced by the previous ablation operations, for example, a tooth cavity, is subsequently to be filled in a way known per se with a material for which the shape of the cavity has to be determined. Conventional and in part very expensive methods of dentistry can be replaced when use is made of a photosensitizer covering the cavity surface and it is scanned with the diagnostic radiation so that the topography of the cavity can be determined via the received light pulses and the transit times. It may be pointed out that there is to be seen in such a method an independent invention that can be applied independently of the aspects chiefly described here, but which can optionally be combined at the discretion of the person skilled in the art with all the aspects and features described in this application.

In accordance with a further embodiment, it can be provided that the region to be processed is determined during diagnosis without the use of a marker or photosensitizer, specifically by determining the presence of a signal generated in the tissue and, if appropriate, the signal strength thereof. In this case, the signal can be the second or a higher harmonic of an electromagnetic radiation irradiated onto the tissue. The electromagnetic radiation can be that of a diagnostic laser beam that has an energy density on the surface of the tissue that is smaller than the energy density that is required for processing the tissue. The processing laser beam and the diagnostic laser beam can be produced by one and the same laser radiation source. Particularly for the purpose of distinguishing between undamaged tooth material and carious tooth material, the tissue can be excited by the diagnostic laser beam by means of the LIBS (Laser Induced Breakdown Spectroscopy) method in the infrared region, a backscattered signal of a second harmonic indicating a healthy tissue (for example, mineralizable collagen fibers), and the absence of such a signal indicating a carious tooth material (that is to say irreversibly damaged=non-mineralizable collagen structures). A region of the tissue can be scanned with the diagnostic laser beam, and the data of the backscattered second harmonic can be detected and stored, and it is then possible to use these data to determine in which sections of the region the processing or ablation is to be performed by the processing laser beam.

In accordance with a further aspect of the present invention, a laser processing device may be specified for processing biological tissue according to the previously described method, the laser processing device having a laser radiation source for providing a pulsed processing laser beam, a laser beam decoupling unit to decouple the laser beam in the direction of a region of the tissue to be processed, and an output device for outputting a photosensitizer in the direction of an area surrounding the region of the tissue to be processed, the output device particularly being connected to the laser beam decoupling unit.

In accordance with one embodiment of the laser processing device, it can be provided that the laser radiation source is provided by a laser oscillator whose output pulses can be fed to the decoupling unit without further amplification so that—as already set forth above—the laser radiation source can be designed cost-effectively. It can prove to be necessary, in this case, to focus the laser beam more intensely so as to be able to reach the required energy densities. However it is also possible to use a laser radiation source consisting of a laser oscillator and an optical amplifier of the laser pulses, in which case conventional focusing can prove adequate.

In accordance with one embodiment of the laser processing device, the wavelength of the laser pulses and the photosensitizer are selected in such a way that the processing laser beam is absorbed at least partially by a single photon absorption in the photosensitizer, the absorption being in the vicinity of an absorption maximum of the photosensitizer.

In accordance with one embodiment of the laser processing device, the wavelength of the laser pulses and the photosensitizer are selected in such a way that the processing laser beam is absorbed at least partially in the photosensitizer by an N photon absorption, with N≧2, the absorption being in the vicinity of an absorption maximum of the photosensitizer.

In accordance with one embodiment, the laser pulses have a half value width (FWHM, full width at half maximum) in a range between 100 fs and 1 ns, this range also encompassing each incremental intermediate value and the increment being 100 fs.

In accordance with one embodiment, the wavelength of the laser pulses lies in a range between 100 nm and 10.6 μm, this range also encompassing every incremental intermediate value, and the increment being 100 nm. This range therefore also encompasses, for example, the output wavelengths of Er:YSGG (λ=2.7 μm) or Er:YAG lasers (λ=2.94 μm) whose wavelengths lie in the infrared wavelength region, or a CO₂ laser, whose output wavelength lies at 10.6 μm. All wavelengths in the specified range are conceivable when combined with suitable existing photosensitizers, or ones yet to be developed.

In accordance with one embodiment, the energy density of the laser pulses on the surface of the tissue and/or the photosensitizer to be processed lies in a range below 7.5 J/cm² per pulse. Depending on the selection of the photosensitizer, an energy density of 1.0 J/cm² per pulse or else therebelow can be adequate for the ablation.

In accordance with one embodiment, the energy of the laser pulses lies in a range below 100 μJ, in particular a focus of the processing laser beam on a surface of the tissue being set to a focal diameter in a range between 1 μm and 100 μm. The range of 1 μm to 5 μm can be achieved with the aid of special optics.

In accordance with one embodiment, a repetition rate of the laser pulses is set in a range between 1 Hz and 10 MHz.

In accordance with one embodiment, the laser processing device is designed as a dental laser processing device for the ablation or removal of tooth material, in particular carious tooth material.

In accordance with one embodiment, the laser processing device further comprises a fixing means connected to the laser beam decoupling unit to spatially fix a distal end of the laser beam decoupling unit with reference to a section of the tissue to be processed.

In accordance with one embodiment, the laser handpiece comprises an attachment that serves not only as fixing means but also sheaths the tooth in such a way that it is possible together with an integrated suction channel system to generate various chemical compositions and/or various pressure conditions by means of which even mercury, for example in the form of an amalgam filling, can be ablated.

In accordance with one embodiment, the laser processing device further comprises a beam shaping unit to shape a substantially rectangular beam profile of the pulsed processing laser beam.

In accordance with one embodiment, the laser processing device further comprises a scanning unit to scan a region of the tissue with the processing laser beam or the diagnostic laser beam. In this case, the scanning unit can, in particular, be designed in such a way that exactly one laser pulse is applied to a sub-region covered by the focus of the processing laser beam. For example, the scanning unit can be designed in such a way that mutually adjacent sub regions covered by one laser pulse in each case have a spatial overlap with one another whose surface area is smaller than half a sub-region.

In accordance with one embodiment, the laser processing device further comprises an autofocus unit to keep constant the spatial position of the focus on the surface of the tissue.

In accordance with one embodiment, the laser processing device further comprises a detection unit to detect the presence of a signal generated in the tissue or in an area surrounding it, and the strength of said signal, if appropriate. The detection unit can have an optical sensor that is, for example, designed to detect a second or higher harmonic of an electromagnetic radiation irradiated onto the tissue. The electromagnetic radiation can be that of a diagnostic laser beam which is produced by one and the same laser radiation source like the processing laser beam, or it can be that of an incoherent and, in particular, continuously emitting light source such as a light-emitting diode (LED).

In accordance with one embodiment, the laser processing device can have a control unit that is designed for the purpose of setting the laser radiation source to a processing mode or a diagnostic mode, the pulsed processing laser beam being produced in the processing mode, and there being produced in the diagnostic mode a diagnostic laser beam that has an energy density on the surface of the tissue which is smaller than the energy density that is required for processing the tissue. In principle, this can be the same laser beam as the processing laser beam whose energy has merely been changed by the control unit in such a way that the energy density is smaller than the energy density required for the processing. If appropriate, instead of or in addition to the energy it is also possible to change the focusing so that a lower energy density results.

In accordance with one embodiment, the laser beam decoupling unit can be provided in the form of a handpiece, it being possible to arrange the output device for outputting the photosensitizer at least partly inside the handpiece, in particular one end of a supply line with a nozzle possibly being arranged in the handpiece. The scanning unit and/or the autofocus unit can also be arranged inside the handpiece.

In accordance with one embodiment, the laser processing device further comprises a further output device for outputting a marker, it being possible, in particular, likewise to arrange the further output device partly inside the handpiece.

In accordance with a further aspect of the present invention, a method for processing biological tissue and a corresponding laser processing device without the use of a photosensitizer are specified, the method comprising the provision of a pulsed processing laser beam and the processing of the tissue by irradiation with the pulsed processing laser beam, and the pulsed processing laser beam being such that the wavelength of the laser pulses lies in a range between 100 nm and 10.6 μm, the temporal half value width (FWHM) of the laser pulses lies in a range between 100 fs and 1 ns, and the energy density of the laser pulses lies below 7.5 J/cm².

In accordance with one embodiment, the energy density of the laser pulses is 1.5 J/cm² or less or 1.0 J/cm² or less.

In accordance with one embodiment, a laser beam focus with a focal diameter of 1 μm to 100 μm is produced on the surface of the tissue. The range of 1 μm to 5 μm can be achieved with the aid of special optics.

In accordance with one embodiment, the laser radiation source for carrying out this method includes a laser oscillator, and the laser pulses generated by the laser oscillator are used for processing the tissue without further optical amplification, that is to say are fed to the decoupling unit in particular without further optical amplification.

This general and specific description can be implemented using an apparatus, a method, a system, or any combination of those. The details of one or more implementations are set forth in the accompanying drawings and the description below. Further, features, aspect, and advantages will become apparent from the description, the drawings, and the claims.

### BRIEF DESCRIPTION OF THE DRAWINGS

The embodiments are explained in more detail below in an exemplary way with the aid of figures of which:

FIG. 1 is a diagrammatic illustration of one embodiment of a laser processing device;

FIG. 2 is a diagrammatic illustration of another embodiment of a laser processing device;

FIG. 3 is a diagrammatic illustration of one embodiment of a laser beam decoupling unit;

FIG. 4 is a diagrammatic illustration of another embodiment of a laser beam decoupling unit;

FIG. 5 is a diagrammatic illustration of another embodiment of a laser beam decoupling unit;

FIG. 6 is a diagrammatic illustration of another embodiment of a laser beam decoupling unit;

FIG. 7 is a diagrammatic illustration of another embodiment of a laser processing device;

FIG. 8 is a flow chart of one example of an automated combination diagnostic and lasering method; and

FIG. 9 is a flow chart of another example of an automated combination diagnostic and lasering method.

### DESCRIPTION OF EMBODIMENTS

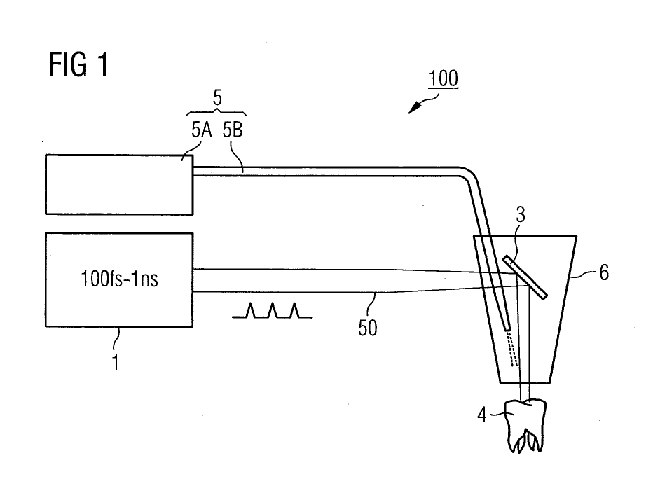

FIG. 1 illustrates one embodiment of a laser processing device for processing biological tissue, but not to true scale. FIG. 1 shows a dental laser processing device for processing, abrading or ablating dentin, particularly carious dentin. However, the laser processing device may be any other kind of medical laser processing device for processing some other kind of biological tissue. For example, ophthalmology can be another potential field of application.

The laser processing device 100 comprises a laser radiation source 1 that may emit a pulsed laser beam 50 with a laser pulse ranging between 100 fs and 1 ns (Full Width Half Maximum). The laser beam may be focused on a patient's tooth 4. It may be necessary to first deflect the laser beam with an optical deflection unit 3 such as a mirror or deviation prism.

The laser radiation source 1 may generate the laser pulses so that the energy per pulse does not exceed 100 μJ. In this case, the focusing of the laser beam can be set in such a way that the processing laser beam 50 on the surface of the tooth 4 has a focus with a focal diameter in a range from 1 μm to 100 μm. The laser radiation source 1 may emit the laser pulses with a repetition rate range from 1 Hz to 10 MHz.

The laser processing device 100 further comprises an output device 5 to output a photosensitizer in the direction of the tooth 4. As shown in FIG. 1, the output device 5 may include a storage chamber 5A to house the photosensitizer where the storage chamber 5A may be connected to a supply line. The photosensitizer may be erythrosine which can be efficiently activated by two-photon absorption of the laser beam of a Nd:YAG laser (1064 nm) or by one-photon absorption of the frequency doubling component of the Nd:YAG laser (532 nm). For example, photosensitizers may be methylene blue, photofrine, or metalorganic dendrimeres. It is understood that all other photosensitizers referenced in technical literatures even if those are still yet to be developed, may be used if the photosensitizer requires the laser wavelength to be adapted to the corresponding maximum absorption of the photosensitizer or at least in the ambience thereof. The photosensitizers may also be biochemical chromophors. The term photosensitizer may also cover such substances which are not photosensitizers by definition but which may feature properties typical for photosensitizers under defined physical-chemical conditions. Some examples may be any gases, gas mixtures (air), or aerosols if those substances feature photosensitizer properties under defined physical-chemical conditions.

The laser processing device 100 may also comprise a decoupling unit 6 for decoupling the laser beam 50 in the direction of the tooth 4. As shown in FIG. 1 as an example, the decoupling unit 6 may contain a deflection unit 3 and may be connected with the supply line of the output device 5 so that the photosensitizer, when being applied, may be jetted towards the tooth 4 from the distal end of the decoupling unit 6 from the supply line.

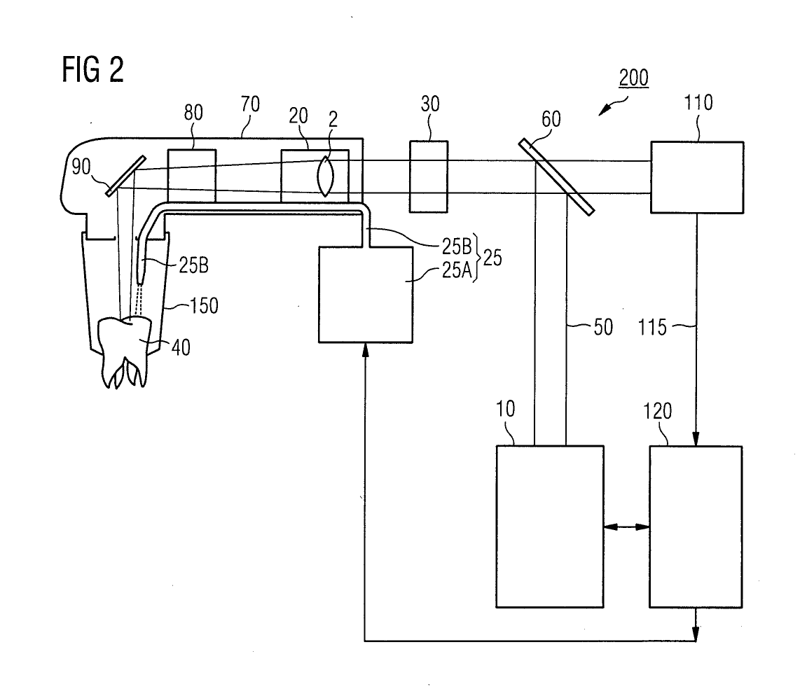

FIG. 2 illustrates another embodiment of a laser processing device, but not to true scale. The embodiment of a laser processing device 200 shown in FIG. 2 comprises a laser radiation source 10 that may emit a pulsed laser beam 50. In this exemplary embodiment, the laser radiation source 10 may be a Nd:YAG laser coupled to a transient or regenerative amplifier emitting laser pulses in a wavelength of 1064 nm. Any other laser radiation source such as a Nd:YVO4 or Nd:GdVO4 laser may be used. The pulse duration of the laser pulses may be 10 ps, the repetition rate may range from 1 kHz to 1000 kHz, the energy of the laser pulses may amount to 40 μJ, and whilst at a repetition rate of 100 kHz the mean beam power may be 4 W.

Any other laser may be used as the laser beam source. For example, a diode laser or a diode laser array may be used. For example, the laser radiation source can be a diode laser or a diode laser array that can be accommodated in the handpiece in a particularly compact way. In particular, the output pulses of the laser radiation source can be used without further optical amplification. That is to say they can be fed to the handpiece or the decoupling unit.

In the embodiment shown in FIG. 2, the laser beam 50 emitted from the laser radiation source 10 may be directed at an optical deflection unit 60 which may selectively divert the laser beam 50 at about 90° at the required wavelength of the laser beam 50. Diverted laser beam 50 may pass through a beam shaping unit 30 to generate a substantially rectangular or a top hat beam profile.

Then, the laser beam 50 may enter a decoupling unit configured as a handpiece fronted by a lens 2 as part of an autofocus unit 20, which may ensure that the focal position created by the lens 2 always remains within the plane of the surface of the tooth 40 to be irradiated. The autofocus unit 20 may be combined with an optical sensing means that senses backscattered radiation from the surface of the tooth 40 to sense whether the surface is still in the focal position of the laser beam. If it is not, a control signal is communicated to the autofocus unit 20 for the laser beam to suitably result on the surface of the tooth 40 and return into the focal position of the laser beam by moving the lens 2 forwards or backwards along the propagation path of the laser beam 50. The lens 2 may be moved by a fast stepper motor connected to a carriage mounting the lens 2. However, it is just as possible to configure the lens 2 for its refraction to be tweaked.

FIG. 2 also illustrates how the lens 2 may be arranged so that it focuses the beam on the surface of the tooth 40 with a focal diameter of 40 μm. The laser pulse energy as recited above may result in an energy density of 3.18 J/cm² which may produce a pulse peak intensity of 3.18·10¹¹W/cm² corresponding to a photon flux density of 1.7·10³° photons·cm²·s⁻¹. The electric field strength of the alternating electromagnetic field may be 1.55·10⁷ V/cm and the median electron oscillation energy in the alternating electromagnetic field may amount to 0.021 eV.

It is understood that the beam shaping unit 30 may also be located in the beam path downstream of the lens 2, particularly in the handpiece 70 although it is just as possible to combine the autofocus unit 20 and beam shaping unit 30, especially the lens 2 and beam shaping unit 30 into a common optical component.

The decoupling unit may also include a scanning unit 80 that may scan over a defined region of the surface of the tooth 40 with the laser beam 50 or a diagnostic laser beam by two rotating mirrors, each facing the other. Also, a deflection unit 90 such as a diverting prism or a reflective mirror may be included to divert the laser beam 50 or a diagnostic laser beam in the direction of the tooth 40.

It is understood that although the scanning unit 80 is arranged in the handpiece in this embodiment, other embodiments may locate the scanning unit in the beam path upstream of the handpiece, i.e. particularly within an arm hinging the mirror or at the input thereto upstream of the handpiece.

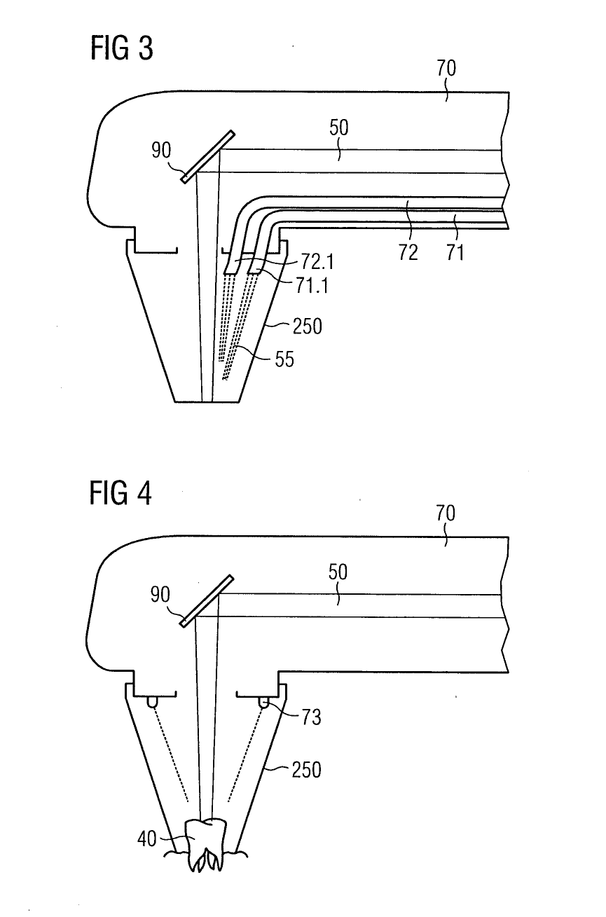

The decoupling unit configured as a handpiece may need to be held directed on the tooth being irradiated by the physician. In maintaining the position of the distal end of the decoupling unit constant relative to the tooth 40, a funnel-shaped fixing element 150 is secured to the distal end of the decoupling unit and can be suitably located on the tooth 40 during lasering as illustrated in FIGS. 3 to 6. A cofferdam or rubber clamp may be placed by the physician to encapsulate the tooth in isolating it from the remaining pharyngeal space.

The laser processing device 200 may further comprise an output device 25 for outputting a photosensitizer in the direction of the tooth 40. The output device 25 may contain a storage chamber 25A that is connected to a supply hose 25B. The supply hose 25B may be ported into the decoupling unit and guided within the decoupling unit into the fixing element 150.

The optical or acoustical signals generated from the irradiated region of the tooth 40 surface or from the ambience thereof can be detected and used for diagnostic purposes. As explained already, the optical signals may be based, for example, either on the plasma radiation or second harmonic generated (SHG) or higher harmonic generated electromagnetic radiation acting on the dentin involved in lasering. The exemplary aspect as shown in FIG. 2 will now be explained with an example of detecting a SHG signal.

In this mode of diagnosis, a diagnostic laser beam may be emitted like the lasering beam is pulsed for diagnosing whether the sub-region of the dentin is carious or not. Here, the energy or energy density is below the threshold for generating ablation or plasma so that no lasering occurs with the diagnostic laser beam. If not, energy or energy density furnishes a higher SHG signal than carious dentin.

At least some part of the radiation having doubled frequency and being generated from the tooth surface may pass through the laser beam path in the opposite direction, as described above. In other words, the radiation may be diverted by the deflection unit 90 and pass through the scanning unit 80 and the autofocus unit 20 with the lens 2 to finally incident the optical deflection unit 60, such as a beam splitter. Here, the beam splitter may be transparent for the wavelength of the SHG signal so that the frequency doubled radiation can be input in an detector 110. The detector 110 may be a simple photo detector detecting the intensity of the SHG radiation. It is just as possible to use a more complex system such as a spectrometer, CCD camera, or CMOS image sensor as the detector 110. Such detectors may suitably be used in combination with the autofocus unit 20, as already indicated above.

Likewise, the deflection unit 90 may be engineered to transmit the frequency-doubled radiation generated from the tooth surface and to direct the radiation to the detector 110 with, for example, a glass fiber located downstream of the deflection unit 90. This may reduce the complexity of the optical beam in transmitting the frequency-doubled radiation since the optics 80, 20, 60, 2 are not designed for several different wavelengths, making them to be coated if necessary. In order to effectively couple the frequency-doubled light, an optical component can be inserted between the deflection unit 90 and the glass fiber to focus the frequency-doubled light onto the glass fiber. This optical component can be engineered as a microoptical component.

The SHG radiation values detected by the detector 110 are converted into a signal 115 and transmitted into a combined analyzer/controller 120, which may also be a computer system for this embodiment. In principle, any other type of control system may be compatible, for instance, memory-programmable controllers, micro controllers, or analog closed-loop controls.

The analyzer/controller 120 can receive a signal containing data as to the operation status of the analyzer/controller 120 from the laser radiation source 10. The analyzer/controller may output a control signal to the laser radiation source 10 in switching the laser radiation source 10, for example, from an standby mode to a operating mode. Here, the analyzer/controller may function upon receiving the signal 115 communicated by the detector 110.

The embodiment shown in FIG. 2 may comprise a laser radiation source 10 which is nimble in mode switching “out” (standby mode), “diagnostics”, and “therapy” (processing) treatment. In this embodiment, the laser radiation source 10 may emit both the laser beam required during the “therapy” mode and the diagnostic laser beam required during the “diagnosis” mode with a substantially different energy density per pulse applied to the tooth in W/cm². Here, the energy density applied to the surface of the tooth needs to be reliably below the ablation threshold in the “diagnosis” mode while the energy density is above this threshold in the “therapy” mode.

In a diagnostic mode as described above, a certain surface region of the tooth 40 is scanned with the diagnostic laser beam and the backscattered SHG signal is received and analyzed. This may allow the surface region can be mapped to a certain extent in identifying a portion of the surface to be irradiated or ablated. As implementing the diagnostic mode, the analyzer/controller 120 may output a signal to the output device 25 and this signal may allow the supply hose 25B and end portion of the controllable nozzle to jet the photosensitizer towards the portion of the tooth surface to be ablated.

FIG. 3 illustrates another example embodiment for a diagnosis. The embodiment illustrated in FIG. 3 includes a decoupling unit in the form of a handpiece shown in cross-section. With this particular embodiment, healthy dentin may be distinguished from unhealthy one by means of a marker rather than using a SHG signal. The mark may indicate a characteristic stain when it is in contact with the unhealthy dentin. This marker can be applied to the tissue via a supply hose 72 that may also be incorporated within the handpiece as shown in FIG. 3. Once the carious portions of a tissue surface are detected preferably by means of optical imaging with subsequent analysis thereof, photosensitizer is applied to these portions via the supply hose 71 for subsequent ablation by the laser beam 50. Accordingly, in this example embodiment, there is no diagnostic laser beam, switching of the laser beam source, or SHG detection. The two supply hoses 71 and 72 can be used to connect the nozzles 71.1 and 72.1 respectively for a controlled orientation in jetting the materials pin-pointed to the surface of the tissue.

It is to be noted that the embodiment as shown in FIG. 3 may depict a laser beam decoupling unit as a stand-alone embodiment. This laser beam decoupling unit may comprise a handpiece 70, a deflection unit 90 for deflecting a lasering beam 50 and/or a diagnostic laser beam, and an attachment 250 for locating the handpiece 70 on an ambience of the tissue to be irradiated. In this arrangement, the handpiece 70 may be configured so that a photosensitizer can be applied via the supply hose 71 incorporated in the handpiece 70 and, where necessary, marker can be jetted via additional supply hose 72 on a portion of the tissue to be irradiated or diagnosed. It is understood that this separate embodiment can also be combined with any of the other embodiments as described in this application and/or sophisticated with any of the features cited in this application, including also leading devices such as a laser processing device incorporating a laser beam decoupling unit as described above.

Referring now to FIG. 4, a decoupling unit in the form of a handpiece, shown in cross-section, illustrates another example embodiment. Here, at least one LED 73 is integrated within the handpiece 70. As shown in the embodiment of FIG. 4, several LEDs 73 may also be incorporated within the handpiece 70 that may serve a physician to illuminate the pharyngeal space when the attachment 250 is still to be affixed in place. This may allow the physician to optimally position the attachment 250 in relation to the tooth 40 being treated. In addition, these LEDs may also serve to activate a marker applied to the surface of the tooth being treated so that the carious locations may indicate a characteristic stain. The image created by the marker in this way can be scanned by the same optics used to incouple the laser beam 50. On the basis of this imaging, the photosensitizer can be applied to the regions to be irradiated or ablated. The LEDs 73 may be arranged on a horizontal end portion of the handpiece 70. For example, The LEDs 73 may be arranged in a circle to achieve illumination as best possible homogenous and rotationally symmetrical. The LEDs 72 may be connected by leads (not shown) integrated within the handpiece 70 for powering the LED 73. The LEDs 73 may be LEDs emitting light in a single color, for example, red, such as quasi-monochromatic LEDs. However, white light LEDs could be used for a better illumination of the pharyngeal space and circumstances so that a larger choice of markers for activation at differing wavelengths is available.

It is to be noted that the embodiment as shown in FIG. 4 may depict a laser beam decoupling unit as a stand-alone embodiment. This laser beam decoupling unit may comprise a handpiece 70, a deflection unit 90 for deflecting a laser beam 50 and/or a diagnostic laser beam, and an attachment 250 for locating the handpiece 70 on an ambience of the tissue to be irradiated. This laser beam decoupling unit may further comprise at least one LED 73 for illuminating and/or activating a marker or photosensitizer. It is understood that this separate embodiment can also be combined with any of the other embodiments as described in this application and/or sophisticated with any of the features cited in this application, including also leading devices such as a laser processing device incorporating a laser beam decoupling unit as described above.

Referring now to FIG. 5, a decoupling unit in the form of a handpiece 70, shown in cross-section, illustrates another example embodiment. Here, the handpiece 70 may feature an attachment 350 having an encapsulating function in addition to a locating function of the tooth 40. As illustrated in the exemplary embodiment of FIG. 5, the seal 350.1 may be applied to the bottom rim of the attachment 350. Here, the seal 350.1 is indicated simply symbolically and not necessarily to be appreciated as being technically realistic. One object of such an attachment may be to encapsulate the direct vicinity of the tooth 40 being treated at best air- and gas-tight from the remaining pharyngeal space. Such an encapsulated location of this kind may allow to optimize the treatment of the tooth in a wide variety of ways as will now be explained with the following example aspects. For example, an aspirator may be integrated within the handpiece 70 to allow the attachment to seal off the region from the outside and this may result efficient and reliable removal of the ablated debris. In addition, a controlled atmosphere can be created surrounding the tooth 40.

It is to be noted that the embodiment as shown in FIG. 5 may depict a laser beam decoupling unit as a stand-alone embodiment. This laser beam decoupling unit may comprise a handpiece 70, a deflection unit 90 for deflecting a lasering beam 50 and/or a diagnostic laser beam, and an attachment 350 to locate the handpiece 70 on an ambience of the tissue to be irradiated. In this arrangement, the attachment 350 may be designed to seal and encapsulate a tissue region to be irradiated. It is understood that this separate embodiment can also be combined with any of the other embodiments as described in this application and/or sophisticated with any of the features cited in this application, including also leading devices such as a laser processing device incorporating a laser beam decoupling unit as described above.

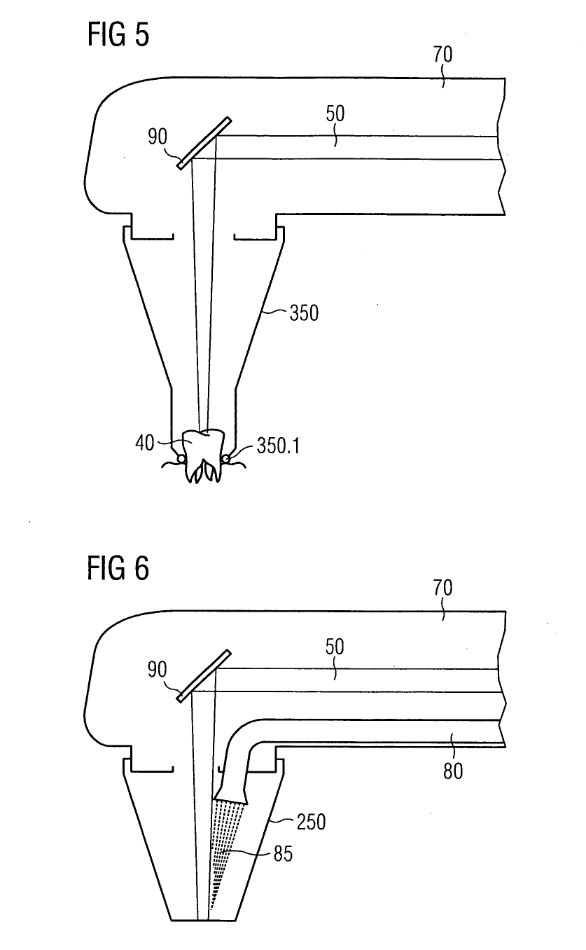

Referring now to FIG. 6, a decoupling unit in the form of a handpiece 70, shown in cross-section, illustrates another exemplary embodiment. Here, the handpiece 70 may mount an attachment 250 and may be configured to integrate an aspirator duct 80 for efficient aspiration of the ablated debris in tissue treatment. The aspirator duct 80 may be connected to an aspirator system (not shown) integrated in the handpiece 70. An open end of the aspirator duct protruding into the attachment 250 such that it is directed at the region being irradiated to aspirate the ablated debris materializing in lasering. The end of the aspirator duct 80 may be mounted movable, for example by user's control and orientation. This also includes varying spacing between the aspirator duct 80 and the region being irradiated.

It is to be noted that the embodiment as shown in FIG. 6 may depict a laser beam decoupling unit as a stand-alone embodiment. This laser beam decoupling unit may comprise a handpiece 70, a deflection unit 90 for deflecting a lasering beam 50 and/or a diagnostic laser beam, and an attachment 250 for locating the handpiece 70 on an ambience of the tissue to be irradiated. Here, the handpiece 70 and attachment 350 may be configured so that an aspirator duct 80 is integrated therein and the end of the duct can be directed at the tissue region being irradiated. It is understood that this separate embodiment can also be combined with any of the other embodiments as described in this application and/or sophisticated with any of the features cited in this application, including also leading devices such as a laser processing device incorporating a laser beam decoupling unit as described above. Especially, a combination of the embodiments as illustrated in FIGS. 5 and 6, i.e. an encapsulated sealed attachment to an aspirator system may allow potentially toxic lasering. For example, the ablation of amalgam fillings can be performed, in which case the gas-tight encapsulation may make it safe to remove the ablated debris, essentially elementary mercury with practically no remainders. As described in this application, laser ablation of the amalgam filling could be performed with the assistance of a photosensitizer. Thus, this embodiment may allow performing amalgam removal by lasering in compliance with the maximum workplace concentration (MAK) as required by law for mercury vapors.

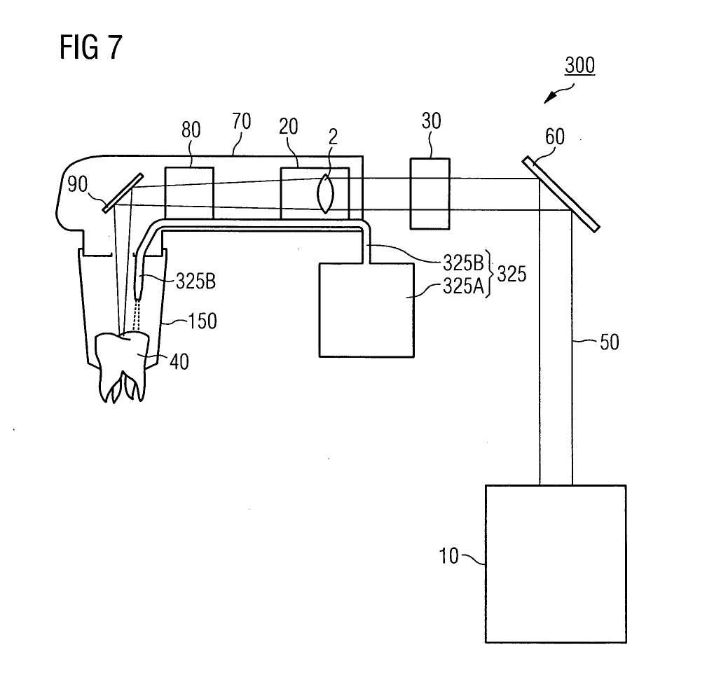

FIG. 7 illustrates a further embodiment of a laser processing device not shown true to scale. The embodiment of a laser processing device 300 shown in FIG. 7 comprises substantially the same components as the components of exemplary embodiment described in FIG. 2 which are identified with the same reference numerals. However, unlike the laser processing device illustrated in FIG. 2, the laser processing device 300 may feature a generator unit 325 comprising a generator unit 325A connected to the handpiece 70 by a line 325B. The line 325B may be integrated through the handpiece to the fixing element 150 and may feature an orifice directed at the tooth being irradiated at the end of the supply hose. Here, the generator unit 325 shown in FIG. 7 does not illustrate its detailed features but the generator unit 325 may have various functions. For example, the generator unit 325 may serve predominantly to create a certain atmosphere in the ambience of the tooth 40 being treated.

In one simple variant, vacuum atmosphere can be generated by the generator unit 325 comprising a vacuum pump. In this example, the fixing element 150, like the attachment 350 of the embodiment described in FIG. 5, may be configured as an encapsulating attachment. In addition, the fixing element 150 may be—when wanted or necessary—sealed off from the handpiece 70 by disposing a window transparent to the lasering beam 50 between the handpiece 70 and the fixing element 150. In a somewhat less complicated variant, when vacuum atmospheres are needed to be created above the tooth 40, there may be no seal or at least none-complete seal provided between the handpiece 70 and the fixing element 150. The generator unit 325 may also be designed to create a positive pressure. Furthermore, the generator unit 325 may be designed to create a specific gas atmosphere in the ambience of the tooth 40 such as furnishing a gas such as O₂, N₂, H₂O (water vapor) or some rare gas. Especially when ablating amalgam fillings, utilizing the generator can be advantageous in binding the ablated mercury in a certain way to remove amalgam fillings from the ambience of the tooth 40. The generator unit 325 may also be designed to cool the tooth 40 by generating a cooling medium by jetting cooling air on to the ablated surface region. The generator unit 325 may also be designed as an aerosol generator that may generate a gas in which particles such as microscopic (nano) or macroscopic particles are dispersed in handling certain functions for the ablation. These particles may have a cooling function. In addition to this, the analyzer/controller 120 and the detector 110 of the embodiment described in FIG. 2 may be included in this particular embodiment described in FIG. 7. Here, the analyzer/controller 120 may also be connected to the generator unit 325 so that the analyzer/controller 120 may control the generator unit 325.

It is to be noted that the embodiment as shown in FIG. 7 may depict a laser processing device as a stand-alone embodiment. This laser processing device may comprise a source 10 to furnish a lasering beam 50, a decoupling unit to decouple the lasering beam 50 in the direction of the tissue region being irradiated, and a generator unit 325 for generating or furnishing an atmosphere in an ambience of the tissue being irradiated. It is understood that this stand-alone embodiment can also be combined with any of the other embodiments as described in this application and/or sophisticated with any of the features cited in this application.

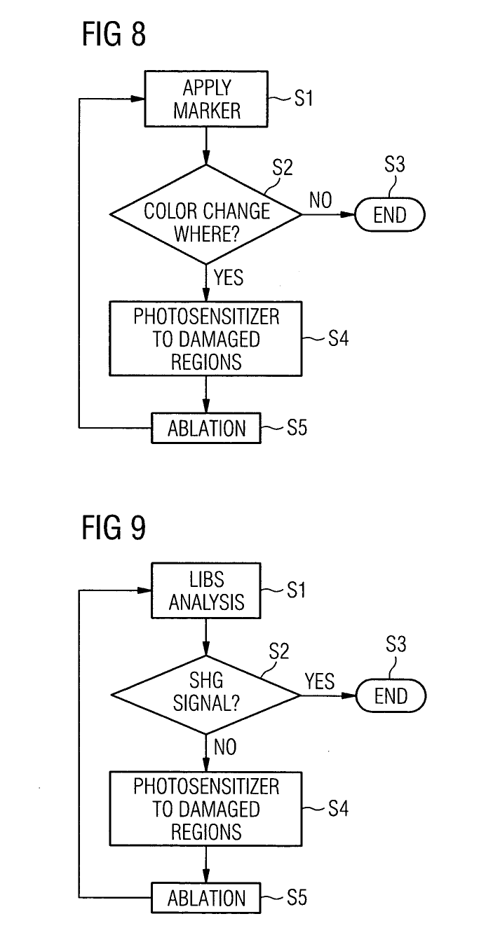

FIG. 8 illustrates a flow chart for one example of methods of an automated combination ablation and diagnostic process when using a marker. In operation S1, a marker may be applied (S1). Then, it is established whether a change in stain has been occurred, indicating damaged tissue (S2). If no change in stain is detected, the process may be discontinued. The changes in stain may be detected with a spatial resolution of the surface being imaged on a detector such as a CCD or CMOS element. Here, the changes may be detected by scanning the image and electronically storing the result of the spatial resolution. Then, the marker may be removed and a photosensitizer may be applied to the regions detected as damaged (S4). The, the ablation may be done by the laser beam (S5). Here, the parameters such as, but not limited to, duration or power of the lasering may be previously set by the user. After this, the process may repeat from S1.

Now, FIG. 9 illustrates a flow chart for one example of methods of an automated combination ablation and diagnostic process using LIBS technology. In operation S1, a region may be scanned with a diagnostic laser beam and simultaneously the detection of a SHG signal may be performed as described for the embodiment illustrated in FIG. 2 (S1). Then, it is established which regions may be viewed as healthy by detecting a backscattered SHG signal from the region. When an SHG signal is returned from all of the surface, the process may be discontinued. Thus, establishing which regions are healthy may be performed with a spatial resolution. Here, the complementary regions can be electronically stored as being diseased and a photosensitizer may be applied to such regions (S4). Then, the ablation is performed with the laser beam (S5). Here, the parameters such as, but not limited to, duration or power of the lasering may be previously set by the user. After this, an LIBS analysis may be repeated from S1.

It is to be noted that the embodiments as shown in FIGS. 8 and 9 may depict a combined lasering and diagnosis process as a stand-alone embodiment. The embodiments may comprise the operations: detecting diseased regions by means of marker or LIBS, applying a photosensitizer to the diseased regions, ablating the diseased regions by means of a laser beam, and repeating detection of any remaining disease and application of photosensitizer until no more disease is detected. It is understood that each of these stand-alone embodiments can also be combined with any of the other embodiments as described herein and/or sophisticated with any of the features cited herein.

It is again to be understood that all features described in the embodiments and stand-alone embodiments may also be applicable to any other embodiments and stand-alone embodiments as described. Also, it may be pointed out that the above embodiments are exemplary, and that the invention disclosure content herein also covers the combinations of features which are described in different exemplary embodiments, to the extent that this is technically possible.

1-3. (canceled)

4. A method for processing a biological tissue, wherein a pulsed laser beam is provided in a processing mode, the method comprising:
irradiating a region of the tissue to be processed with the pulsed processing laser beam,
the laser beam emitting laser pulses with a temporal full width at half maximum in a range between about 100 femtosecond and about 1 nanosecond,
the laser beam having a laser pulse wavelength in a range between about 100 nm and about 10.6 μm, and
the laser beam having an energy density per pulse in a range below 7.5 J/cm².

5. The method of claim 1, wherein a repetition rate of the laser pulses is set in a range between about 1 Hz to about 10 MHz.

6. The method of claim 1, wherein the method is employed for at least one of ablation and removal of a tooth material.

7. The method of claim 1, wherein the laser beam comprises a substantially rectangular beam profile.

8. The method of claim 1, wherein the region of the tissue to be processed is scanned by the laser beam.

9. The method of claim 8, wherein the laser beam lasers at least one sub-region, the sub-region being focused by exactly one laser pulse.

10. The method of claim 9, wherein mutually adjacent sub-regions covered by one laser pulse in each case have a spatial overlap with one another, the spatial overlap having a first surface area smaller than one half of a second surface area of the sub-region.

11. The method of claim 1, wherein irradiating the region of the tissue to be processed further comprises controlling the laser beam so that a spatial position of a laser beam focus remains on a surface of the region of the tissue.

12. The method of claim 1, wherein the region of the tissue to be processed is determined in a diagnostic mode.

13. The method of claim 12, wherein
the region of the tissue to be processed is determined by applying a marker to the tissue, and
the marker assumes a characteristic coloration or exhibits another detectable response when in contact with a specific tissue type.

14. The method of claim 13, wherein
the marker is a photosensitizer identical to the photosensitizer used in the processing mode.

15. The method of claim 1, wherein the region of the tissue to be processed is determined by detecting at least one of a presence and a strength of a signal generated in at least one of the tissue and a marker located in an area surrounding the region of the tissue.

16. The method of claim 15, wherein the signal is at least one of a fluorescence radiation, a second harmonic, and a higher harmonic of an electromagnetic radiation irradiated onto at least one of the tissue and the marker.

17. The method of claim 16, wherein the electromagnetic radiation is a diagnostic laser beam radiation having a first energy density on a surface of the tissue or a second energy density of the photosensitizer or the marker, the second energy density being smaller than the first energy density required to process the tissue.

18. The method of claim 17, wherein the laser beam and the diagnostic laser beam are produced by one laser beam radiation source.

19. The method of claim 18, wherein the electromagnetic radiation is a radiation of an incoherent light source.

20. (canceled)

21. A laser processing device to process a tissue, comprising:
a laser radiation source to provide a pulsed processing laser beam emitting laser pulses;
a decoupling unit to decouple the laser beam towards a region of the tissue to be processed; and
focusing means for focusing the laser beam, the laser beam having
a wavelength of the laser pulses in a range between about 100 nm and about 10.6 μm,
a temporal full width at half maximum of the laser pulses in a range between about 100 fs and about 1 ns, and
an energy density of the laser pulses on a surface of the tissue in a range below 7.5 J/cm² per pulse.

22. The laser processing device of claim 21, wherein the device is a dental laser processing device for at least one of ablation and removal of a tooth material.

23. (canceled)

24. The laser processing device of claim 21, wherein a repetition rate of the laser pulses is set in a range between about 1 Hz to about 10 MHz.

25. The laser processing device of claim 21, wherein
the laser beam has a laser pulse wavelength,
the wavelength and the photosensitizer are selected so that at least part of the laser beam is absorbed by a single photon absorption in the photosensitizer, and
the laser beam is absorbed near to a maximum absorption of the photosensitizer.

26. The laser processing device of claim 21, wherein
the laser beam has a laser pulse wavelength,
the wavelength and the photosensitizer are selected so that at least part of the laser beam is absorbed by an N photon absorption where N is more than or equal to 2, and
the laser beam is absorbed near to a maximum absorption of the photosensitizer.

27. The laser processing device of claim 21, further comprising:
fixing means for spatially fixing a distal end of the laser beam decoupling unit relative to a region of the tissue, the fixing means being connected to the laser beam decoupling unit.

28. The laser processing device of or 21, further comprising a beam shaping unit to shape a substantially rectangular beam profile of the pulsed laser beam.

29. The laser processing device of or 21, further comprising a scanning unit to scan the region of the tissue with the laser beam.

30. The laser processing device of claim 29, wherein the scanning unit is configured so that exactly one laser pulse is applied to a sub-region covered by a focus of the laser beam.

31. The laser processing device of claim 30, wherein the scanning unit is configured so that mutually adjacent sub-regions covered by one laser pulse in each case have a spatial overlap with one another, the spatial overlap having a first surface area smaller than one half of a second surface area of the sub-region.

32. The laser processing laser processing device of claim 21, further comprising an autofocus unit to maintain constant a spatial position of the laser beam on a surface of the tissue.

33. The laser processing device of claim 21, further comprising a detection unit to detect at least one of a presence and a strength of a signal generated in at least one of the tissue and a marker located in the area surrounding the region of the tissue.

34. The laser processing device of claim 33, wherein the detection unit comprises an optical sensor.

35. The laser processing device of claim 34, wherein the optical sensor detects at least one of a fluorescence radiation, a second harmonic, and a higher harmonic of an electromagnetic radiation irradiated onto at least one of the tissue, a marker, and the photosensitizer.

36. The laser processing device of claim 21, wherein
the decoupling unit is provided in a form of a handpiece, and
a portion of the output device is contained in the handpiece.

37. The laser processing device of claim 36, wherein at least one of a scanning unit and an autofocus unit is arranged in the handpiece.

38. The laser processing device of or 21, wherein
the laser radiation source includes a laser oscillator, and
the laser pulses generated by the laser oscillator are fed to the decoupling unit without further optical amplification.
